(19) United States
(12) Patent Application Publication
(10) Pub. No.: US 2004/0009198 A1
(43) Pub. Date: Jan. 15, 2004

(54) Long-lasting cosmetic composition comprising a specific proadhesive material

(76) Inventors: Pascale Bernard, Sucy-En-Brie (FR); Anke Hadasch, Paris (FR); Beatrice Toumi, Verrieres-Le-Buisson (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/332,620

(22) PCT Filed: Jul. 9, 2001
(86) PCT No.: PCT/FR01/02206

(30) Foreign Application Priority Data
Jul. 13, 2000 (FR) 00/09217

(51) Int. Cl. A61K 7/00

(52) U.S. Cl. 424/401

(57) ABSTRACT

The invention concerns a composition, in particular cosmetic and/or physiological, for skin or skin appendage make-up and/or care comprising at least a specific pro-adhesive material. Said composition exhibits remarkable non-transfer and/or long lasting properties. The invention also concerns the use of a pro-adhesive material in such a composition.

The present invention relates to a composition comprising a proadhesive material intended in particular for the fields of cosmetics and hygiene. More especially, the invention relates to a long-lasting and transfer-resistant makeup and/or care composition for both human facial skin and body skin and/or for the integuments.

Cosmetic compositions, especially makeup compositions such as lipsticks, foundations, body makeup products, concealer products, eye shadows or powders, generally comprise fatty substances such as oils and/or waxes, and a particulate phase generally composed of fillers and pigments. They may thus be in the form of an anhydrous gel, in the form of a stick or tube or in the form of a soft paste, such as, for example, certain foundations, eye shadows or lipsticks. They may also be in the form of a powder, which may be, for example, free, compacted or pressed. Makeup compositions may also comprise water or a hydrophilic phase, and may then especially be in the form of an oil-in-water emulsion, a water-in-oil emulsion, a multiple emulsion, especially when it is a foundation, a tinted cream, a care cream or an antisun product. Nail varnishes are generally in the form of a solution of an organic solvent.

These compositions do not always show good staying power when they are applied to the skin, mucous membranes or semimucous membranes. Specifically, it has been found that certain compositions have a tendency to travel within the wrinkles and/or fine lines of the skin, in the case of foundations; in the fine lines around the lips, in the case of lipsticks; in the folds of the eyelids, in the case of eye shadows. The appearance of lines in the makeup generated by the movements of the eyelids has also been found, especially in the case of eye shadows. Similarly, the original color is liable to change over time. In the case of nail varnishes, the product is liable to crack, flake or not withstand friction.

Moreover, certain makeup products may have the drawback of transferring. This means that the composition is liable to become at least partially deposited onto certain supports with which it comes into contact, such as, for example, a glass, an item of clothing or the skin.

On becoming deposited, said composition leaves a mark on said support. This therefore results in mediocre persistence of the composition on the skin or mucous membranes, as a result of which it is necessary to renew its application regularly.

Moreover, the appearance of unacceptable traces on certain items of clothing, and especially on blouse collars, may put certain women off using this type of makeup.

These phenomena all result in an unsightly effect that it would obviously be desirable to avoid.

There is thus still a need for products, especially makeup products, that have good staying power while having good cosmetic properties, especially properties of sliding on application, and also softness, moisturization and comfort qualities after applying the makeup.

The Applicant has found, entirely surprisingly, that the use of a particular proadhesive material in a cosmetic or physiological composition produces a makeup of very good staying power, which does not transfer at all and is water-resistant, the composition moreover being easy to break down.

A first subject of the present invention is thus a pulverulent cosmetic and/or physiological composition comprising at least one pulverulent compound and at least one proadhesive material satisfying the following condition:

10⁸ Pa≧G′ (35° C.)≧2×10⁴ Pa for any frequency ranging from 2×10⁻² to 2 Hz, preferably:

10⁷ Pa≧G′ (35° C.)≧2×10⁴ Pa for any frequency ranging from 2×10⁻² to 2 Hz,

in which:

G′ (35° C.) is the elastic shear modulus of said proadhesive material, measured at a temperature of 35° C.

The composition according to the invention has excellent staying power properties on the skin and excellent “transfer-resistance” properties. It adheres well to the skin. It is resistant to friction, sweat, sebum and water. It breaks down easily. It allows a homogeneous deposition over time on the skin and a uniform makeup. Once applied to the skin, it does not migrate and has very good color fastness over time. The composition spreads easily and is very comfortable to wear. It is pleasant to apply and to wear throughout the day. It is not greasy and does not shine over time. It has a pleasant, creamy feel.

Another subject of the invention is the use, in or for the manufacture of a pulverulent cosmetic or physiological composition, of at least one proadhesive material as defined above to reduce, or even eliminate, the transfer and/or migration, and/or to increase the staying power, of the composition applied onto human lips and/or skin onto a support placed in contact with the deposit.

Another subject of the invention is the use, in or for the manufacture of a pulverulent cosmetic or physiological composition, of at least one proadhesive material as defined above to increase the color fastness of the composition over time.

A subject of the invention is also a nontherapeutic care and/or makeup process for human skin, mucous membranes, semimucous membranes and/or integuments, which consists in applying to said human skin, mucous membranes, semimucous membranes and/or integuments a pulverulent cosmetic composition as defined above.

According to the invention, the expression “pulverulent composition or composition in pulverulent form” means a composition that comprises at least one pulverulent compound. The pulverulent compounds may be chosen from pigments and/or nacres and/or fillers and/or mixtures thereof usually used in cosmetic compositions. Advantageously, the pulverulent compounds are present in a content ranging from 60% to 99.9% and more preferably from 80% to 99.9% by weight, relative to the total weight of the composition.

According to the invention, the expression “physiological composition” means a nontoxic physiologically acceptable composition that may be applied to human skin, integuments or lips. According to the invention, the expression “cosmetic composition” means a composition of pleasant appearance, odor and feel.

In one particular embodiment of the invention, the composition according to the invention comprises at least 80% of pulverulent compounds. This produces a product that has a particular texture, in the form of a gum, i.e. a solid product, which does not become deformed under its own weight.

The composition according to the invention also comprises at least one proadhesive material. For the purposes of the present invention, the term “material” means a polymer or a polymer system, preferably at least partially noncrosslinked, even more preferably virtually noncrosslinked, which may comprise one or more polymers of different nature. For the purpose of the present invention, the term “proadhesive” means capable of becoming adhesive generally by interaction with a combined diffusing compound or by interaction with biological exudates. For the purposes of the present invention, the term “adhesive” means a substance that adheres to a surface, i.e., after contact between the index finger and the surface of the composition containing the material, there is a sensation of stickiness of the surface when contact is broken therewith by removing the finger, in the same way as may be experienced after contact with an adhesive surface, for example an adhesive tape. After contact with the skin, the composition comprising the proadhesive material will generally stick to the surface of the skin and will thus have good staying power, or will adhere to a “combined” composition comprising at least one diffusing compound, deposited before and/or after the composition according to the invention; thus, the resulting cosmetic product on the skin will generally not have any surface stickiness, i.e., after contact between the index finger and the surface of the product, there is no sensation of stickiness of the surface when contact therewith is broken by removing the finger.

The proadhesive material according to the invention may optionally have a certain level of bonding power when used alone. After applying the composition according to the invention to the skin, in particular to the face, a composition that has very good staying power is obtained, by virtue of interactions with the biological exudates, which may change over time. The biological exudates are generally physiological fluids and cutaneous excretions, especially sebum, sweat, tears and saliva. It is known to those skilled in the art that, depending on the anatomical location, the levels and nature of these excretions vary in the course of the day.

The proadhesive material may also be used in combination with a diffusing compound which, after diffusion into the polymer network of the proadhesive material, develops bonding power superior to that of the initial material: a “proadhesive material+diffusing compound” system which has adhesive properties is thus obtained. The diffusing compound may be, for example, sebum or sweat secreted by the skin over time. A composition comprising the proadhesive material according to the invention will thus increase in staying power over time with the release of sebum or sweat by the skin.

In one embodiment of the invention, the diffusing compound is present in a second composition, referred to hereinbelow and throughout the present patent application as the “combined composition”, which is applied separately and in combination with the composition according to the invention comprising the proadhesive material. The combined composition may be applied, without distinction, to the skin and/or the integuments before or after the composition according to the invention.

A subject of the present invention is thus also a makeup or care kit comprising:

i) a “combinable” composition comprising at least one proadhesive material that satisfies the following condition:

10⁸ Pa≧G′ (35° C.)≧2×10⁴ Pa for any frequency ranging from 2×10⁻² to 2 Hz, preferably:

10⁷ Pa≧G′ (35° C.)≧2×10⁴ Pa for any frequency ranging from 2×10⁻² to 2 Hz,

in which:

G′ (35° C.) is the elastic shear modulus of said proadhesive material, measured at a temperature of 35° C., and

ii) a “combined composition” comprising at least one diffusing compound such that the “proadhesive material+diffusing compound” adhesive system satisfies the following conditions:

G′_{AS}(2 Hz, 35° C.)≧10³ Pa

10⁸ Pa≧G′_{AS}(35° C.), preferably 10⁷ Pa≧G′_{AS}(35° C.), and

G′_{AS}(2×10⁻² Hz, 35° C.)≧3×10⁵ Pa, in which:

G′_{AS}(2 Hz, 35° C.) is the elastic shear modulus of the adhesive system, measured at a frequency of 2 Hz and at a temperature of 35° C.,

G′_{AS}(35° C.) is the elastic shear modulus of the adhesive system at a temperature of 35° C. (and thus for any frequency within the range between 2×10⁻² and 2 Hz);

G′_{AS} (2×10⁻² Hz, 35° C.) is the elastic shear modulus of the adhesive system, measured at a frequency of 2×10⁻² Hz and at 35° C.

The condition “10⁸ Pa≧G′_{AS}(35° C.), preferably 10⁷ Pa≧G′_{AS}(35° C.)” does not mean that G′_{AS}(35° C.) is constant throughout this frequency range of between 2×10⁻² and 2 Hz, but that, irrespective of the value of the frequency over this range, the condition is satisfied.

For the remainder of the text of the description, the expression “composition according to the invention” means a composition as defined above or a combinable composition as defined above.

The compositions and kits according to the invention especially find a particularly advantageous use in the field of caring for and/or making up the skin, mucous membranes, semimucous membranes and integuments. The term “mucous membrane” especially means the inner part of the lower eyelid; the term “semimucous membrane” more particularly means the lips of the face; the term “integument” means the eyelashes, eyebrows, hair and nails. Thus, the invention finds a most particular application in the field of care and/or makeup products for the lips of the face and for the skin, such as foundations, concealer products, eye shadows, powders, body makeup products, lipsticks, self-tanning products or antisun products, and in the field of care and/or makeup products for integuments (or substitutes thereof, for instance false nails or false eyelashes), such as mascaras and nail varnishes.

The viscoelastic properties of a material are conventionally defined by two characteristic values, which are as follows:

the elastic shear modulus, which represents the elastic behavior of the material for a given frequency and which is conventionally written as G′,

the viscose shear modulus, which represents the viscous behavior of the material for a given frequency, and which is conventionally written as G″.

These magnitudes are especially defined in the “Handbook of Pressure Sensitive Adhesive Technology” 3rd Edition, D. Satas, Chapter 9, pp. 155 to 157.

These viscoelastic properties are measured during dynamic tests under sinusoidal stresses of low amplitude (small deformations) performed at 35° C. over a frequency range of from 2×10⁻² to 20 Hz on a rheometer (for example a Haake RS50 or RS75 rheometer) under a torsional/shear stress, for example in cone-plate geometry (for example with a cone angle of 1°).

When the materials are too cohesive for a person skilled in the art to be able to characterize them with the above Haake RS50 or RS75 rheometer (for example when G′_{AS}(2×10⁻² Hz, 35° C.)>10⁸ Pa), the viscoelastic properties of the materials are measured during dynamic tests under sinusoidal stresses of low amplitude (small deformations) performed at 35° C. over a frequency range of from 2×10⁻² to 20 Hz on a viscoelasticimeter, for example of the type DMA 2980 from T. A. Instruments in traction on film. To do this, the materials are dissolved in a suitable volatile solvent (for example at a concentration of 10%) and then poured in the form of a film into a Teflon-coated matrix; they are then dried at 35° C. in order for the volatile solvent to evaporate off and for a film formed from these materials to be recovered. In this case, the elastic tensile modulus E′ and the viscous tensile modulus E″ are determined and the values G′ and G″ are deduced by means of the relationships G′=E′/3 and G″ E″/3.

The proadhesive materials that may be used according to the present invention satisfy the following condition:

10⁸ Pa≧G′ (35° C.)≧2×10⁴, preferably 10⁷ Pa≧G′ (35° C.)≧2×10⁴, for any frequency ranging from 2×10⁻² to 2 Hz,

in which:

G′ (35° C.) is the elastic shear modulus of said proadhesive material, measured at a temperature of 35° C.

The proadhesive materials according to the invention may be chosen from adhesives of “pressure-sensitive adhesive” type for example, such as those mentioned in the “Handbook of Pressure Sensitive Adhesive Technology” 3rd Edition, D. Satas.

The proadhesive materials according to the invention are preferably adhesive polymers chosen from block and random copolymers comprising at least one monomer or a combination of monomers whose resultant polymer has a glass transition temperature of less than room temperature (25° C.), these monomers or monomer combinations possibly being chosen from butadiene, ethylene, propylene, isoprene, isobutylene and a silicone, and blends thereof. Examples of such materials are block polymers of the styrene-butadiene-styrene, styrene-(ethylene-butylene)-styrene or styrene-isoprene-styrene type, for instance those sold under the trade name “Kraton” from Shell Chemical Co. or “Vector” from Exxon.

The proadhesive materials according to the invention are preferably adhesive polymers chosen from:

polyurethanes,

acrylic polymers,

silicones,

butyl rubbers, especially from polyisobutylenes,

ethylene/vinyl acetate polymers,

polyamides optionally modified with fatty chains,

natural rubbers,

and blends thereof.

In one particularly advantageous form of the invention, the proadhesive materials are chosen from polyisobutylenes with a relative molar mass Mv of greater than or equal to 150,000.

This relative molar mass Mv may be evaluated by its viscosimetric mean, calculated according to the formula J₀=3.06×10⁻² Mv^{0.65}, in which J₀ is the Staudinger index (in cm³/g). This index is calculated from the flow time of a solution with a polyisobutylene concentration of C=0.01 g/cm³ in isooctane, through a capillary I of an Ubbelhode viscometer at 20° C. according to ISO standard 1628.

The relative molar mass Mv may also be evaluated by GPC (gel permeation chromatography) according to the following protocol: 200 μl of a 0.5% solution of polymer (adhesive material) are injected by means of a Waters 6000 A pump, the eluent being a 100% THF solution, flow rate 1 ml/min, at room temperature, through a set of 8 columns: μstyragel 500 Å+10⁴ Å+2×10³ Å+styragel HR0.5+2×HR1+HR5E (300×7.8 mm). The detection is performed on a Waters 410 refractometer and on a Waters 490 UV detector at a wavelength of 254 nm.

As commercial products that are particularly suitable for the present invention, mention may be made of polyisobutylenes with relative molar masses Mv greater than 150,000 of the Oppanol range from BASF, especially the polyisobutylenes with respective relative molar masses Mv of 200,000, 400,000 and 1,110,000 sold under the respective trade names “Oppanol B 30 SF”, “Oppanol B 50 SF” and “Oppanol B 100” by the company BASF, the polyisobutylenes with relative molar masses Mv of between 900,000 and 2,200,000 sold under the trade name “Vistanex MM” by the company Exxon, and blends thereof.

The proadhesive materials are preferably present in the composition according to the invention in a content ranging from 0.1% to 99%, preferably from 0.1% to 30% and more preferably from 0.1% to 10% by weight, relative to the total weight of the composition.

Besides the proadhesive material defined above, the composition according to the invention may comprise any cosmetically acceptable support. The expression “cosmetically acceptable support” means a medium that is compatible with any keratin material such as the skin, the nails, the hair, the eyelashes and eyebrows, the mucous membranes and semimucous membranes and any other area of body or facial skin, and also any substitute for these supports, for instance false nails, false eyelashes and hairpieces.

This support may comprise any cosmetically acceptable water-soluble, water-dispersible, liposoluble or lipodispersible compound conventionally used in cosmetics. Thus, the support may be in the form of a pulverulent composition, of an anhydrous fatty phase (gel or solution), in the form of an aqueous phase (gel or solution), in the form of a dispersion, of a solution in an organic solvent, of an O/W or W/O emulsion, or of a multiple emulsion, optionally stabilized with one or more organized systems.

For the purposes of the present invention, the expression “organized systems” means inverse micelles or “lyotropic liquid crystal” structures which are formed at room temperature by mixing together several surfactants or mixing together surfactants and polar solvents or mixing together several polar solvents, the polar solvents being chosen, for example, from water, glycerol, panthenol, propylene glycol and butylene glycol, and/or mixtures thereof. The liquid crystal state is a state that is intermediate between the solid state and the liquid state. It is often referred to as the mesomorphic state. These organized systems are thermodynamically stable.

Depending on its nature, the proadhesive material according to the invention may be present in the support in dissolved form or in dispersed form, in an aqueous phase or in an anhydrous phase. The proadhesive material according to the invention may thus be in the form of an aqueous dispersion of particles or in the form of an oily dispersion of particles.

When the composition according to the invention comprises a fatty phase, it can preferably comprise at least one cosmetically or physiologically acceptable oil chosen especially from carbon-based, hydrocarbon-based and/or silicone-based oils of mineral, animal, plant or synthetic origin, and mixtures thereof, provided that they are compatible with the intended use.

Mention may also be made of hydrocarbon-based oils such as liquid paraffin or liquid petroleum jelly, mink oil, turtle oil, soybean oil, perhydrosqualene, sweet almond oil, beauty-leaf oil, palm oil, grapeseed oil, sesame seed oil, corn oil, parleam oil, arara oil, rapeseed oil, sunflower oil, cottonseed oil, apricot oil, castor oil, avocado oil, jojoba oil, olive oil or cereal germ oil; esters of lanolic acid, of oleic acid, of lauric acid or of stearic acid; fatty esters, such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate or lactate, 2-diethylhexyl succinate, diisostearyl malate, glyceryl triisostearate or diglyceryl triisostearate; higher fatty acids such as myristic acid, palmitic acid, stearic acid, behenic acid, oleic acid, linoleic acid, linolenic acid or isostearic acid; higher fatty alcohols such as cetanol, stearyl alcohol or oleyl alcohol, linoleyl alcohol or linolenyl alcohol, isostearyl alcohol or octyldodecanol; silicone oils such as polydimethylsiloxanes (PDMS), which are optionally phenylated such as phenyltrimethicones, or optionally substituted with aliphatic and/or aromatic groups, or with functional groups such as hydroxyl, thiol and/or amine groups; polysiloxanes modified with fatty acids, with fatty alcohols or with polyoxyalkylenes, and mixtures thereof.

Advantageously, at least one oil that is volatile at room temperature may be used. The term “volatile oil” means an oil that is capable of evaporating from the skin at room temperature in less than one hour. Preferably, the volatile oil has a viscosity ranging from 0.5 to 25 centistokes at 25° C. After evaporation of these oils, a nonsticky particle deposit is obtained on the skin or mucous membranes. Preferably, oils whose flash point is high enough to allow these oils to be used in the formulation are used. These volatile oils also facilitate the application of the composition to the skin.

These oils may be hydrocarbon-based oils or silicone oils optionally comprising alkyl or alkoxy groups at the end of a silicone chain or pendent.

As volatile silicone oils that may be used in the invention, mention may be made of linear or cyclic silicones containing from 2 to 7 silicon atoms, these silicones optionally comprising alkyl or alkoxy groups containing from 1 to 10 carbon atoms. Mention may thus be made especially of octamethylcyclotetrasiloxane, decamethylcyclopentasiloxane, hexadecamethylcyclohexasiloxane, heptamethylhexyltrisiloxane and heptamethyloctyltrisiloxane, and/or mixtures thereof.

Volatile hydrocarbon-based oils that may be mentioned include C₈-C₁₆ isoparaffins such as isododecane, isodecane, heptane and isohexadecane, and/or mixtures thereof.

These volatile oils may be present in the composition according to the invention in a content ranging from 0.1% to 99% by weight, preferably from 0.1% to 60% and more preferably from 0.1% to 40%, relative to the total weight of the composition. Preferably, the weight ratio of volatile oil to the proadhesive material according to the invention ranges from 1 to 20, more preferably from 1 to 10 and better still from 2 to 4.

The fatty phase may also comprise at least one wax, at least one gum and/or at least one pasty fatty substance, of plant, animal, mineral or synthetic origin, or even silicone-based, and mixtures thereof.

Among the waxes that are solid at room temperature, which may be present in the composition according to the invention, mention may be made of hydrocarbon-based waxes such as beeswax, carnauba wax, candelilla wax, ouricurry wax, Japan wax, cork fiber wax or sugar cane wax, paraffin wax, lignite wax, microcrystalline waxes, lanolin wax, montan wax, ozokerites, polyethylene waxes, the waxes obtained by Fischer-Tropsch synthesis, hydrogenated oils, fatty esters and glycerides that are solid at 25° C. Silicone waxes may also be used, among which mention may be made of alkyl, alkoxy and/or esters of polymethylsiloxane. The waxes may be in the form of stable dispersions of colloidal wax particles as may be prepared according to known methods, such as those of “Microemulsions Theory and Practice”, L. M. Prince Ed., Academic Press (1977), pages 21-32. Mention may be made of jojoba oil as a wax that is liquid at room temperature.

The waxes may be present in a proportion of from 0.1% to 50% by weight relative to the total weight of the composition.

The pasty fatty compounds may be defined by means of at least one of the following physicochemical properties:

a viscosity from 0.1 to 40 Pa.s (1 to 400 poises) and preferably 0.5 to 25 Pa.s, measured at 40° C. with a Contraves TV rotary viscometer equipped with an MS-r3 or MS-r4 rotor at a frequency of 240 rpm,

a melting point of 25-70° C. and preferably 25-55° C.

The compositions of the invention may also comprise at least one alkyl, alkoxy or phenyl dimethicone such as, for example, the product sold under the name “Abil Wax 2440” by the company Goldschmidt.

The compositions according to the invention may also comprise at least one silicone resin comprising a combination of units R₃SiO_{1/2}, R₂SiO_{2/2}, RSiO_{3/2} and SiO_{4/2}, in which R denotes an alkyl radical containing from 1 to 6 carbon atoms.

The fatty phase may also contain at least one liposoluble dye. This liposoluble dye is, for example, Sudan red, DC Red 17, DC Green 6, α-carotene, soybean oil, Sudan brown, DC Yellow 11, DC Violet 2, DC Orange 5, or quinoline yellow, or mixtures thereof. They can represent from 0.01 to 20% of the total weight of the compositions, and better still from 0.1% to 6%.

The fatty phase can represent from 0.01% to 99.9% by weight relative to the total weight of the final composition. When the composition according to the invention is a powder, for example for the face, the fatty phase is preferably present in a content ranging from 0.1% to 30% by weight relative to the total weight of the composition.

The composition according to the invention may comprise at least one organic solvent phase, in particular when the composition is a nail varnish.

As organic solvent that may be used in the invention, mention may be made of:

ketones that are liquid at room temperature, such as methyl ethyl ketone, methyl isobutyl ketone, diisobutyl ketone, isophorone, cyclohexanone or acetone;

alcohols that are liquid at room temperature, such as ethanol, isopropanol, diacetone alcohol, 2-butoxyethanol or cyclohexanol;

glycols that are liquid at room temperature, such as ethylene glycol, propylene glycol, pentylene glycol or glycerol;

propylene glycol ethers that are liquid at room temperature, such as propylene glycol monomethyl ether, propylene glycol monomethyl ether acetate or dipropylene glycol mono-n-butyl ether;

short-chain esters (containing from 3 to 8 carbon atoms in total), such as ethyl acetate, methyl acetate, propyl acetate, n-butyl acetate or isopentyl acetate;

ethers that are liquid at room temperature, such as diethyl ether, dimethyl ether or dichlorodiethyl ether;

alkanes that are liquid at room temperature, such as decane, heptane, dodecane or cyclohexane;

cycloaromatic compounds that are liquid at room temperature, such as toluene and xylene;

aldehydes that are liquid at room temperature, such as benzaldehyde or acetaldehyde,

mixtures thereof.

The organic solvent may be present in the composition of the invention in a content ranging from 0.01% to 99% by weight and preferably from 50% to 99% by weight, for use as a nail varnish, relative to the total weight of the composition.

When the composition according to the invention comprises an aqueous phase, it may comprise water, a floral water such as cornflower water and/or a mineral water such as eau de Vittel, eau de Lucas or eau de La Roche Posay and/or a spring water.

The aqueous phase may also comprise solvents other than water, such as, for example, primary alcohols such as ethanol and isopropanol, glycols such as propylene glycol, butylene glycol, dipropylene glycol or diethylene glycol, glycol ethers such as mono-, di- or tripropylene glycol (C₁-C₄)alkyl ethers, and mono-, di- or triethylene glycol, and mixtures thereof.

The aqueous phase may also comprise at least one water-soluble colorant chosen from the colorants that are common in the field under consideration, such as the disodium salt of ponceau, the disodium salt of alizarin green, quinoline yellow, the trisodium salt of amaranth, the disodium salt of tartrazine, the monosodium salt of rhodamine, the disodium salt of fuchsin, or xanthophyll, and mixtures thereof.

The aqueous phase may also comprise any compound that is compatible with an aqueous phase (water-soluble or water-dispersible), such as gelling agents, film-forming polymers, thickeners, surfactants, preserving agents or pigment dispersions.

When the composition according to the invention is a nail varnish, the aqueous phase may consist essentially of water or of an aqueous-alcoholic mixture especially comprising C₁-C₅ monoalcohols or C₂-C₈ glycols.

Preferably, the aqueous phase is present in the compositions of the invention in a content ranging from 0.01% to 99% by weight and preferably from 0.1% to 80% by weight, relative to the total weight of the composition. When the composition according to the invention is a powder, for example for the face, the aqueous phase is preferably present in a content ranging from 0.01% to 30% by weight relative to the total weight of the composition.

The composition according to the invention may also comprise at least one amphiphilic compound, i.e. a compound comprising both a lipophilic portion (apolar portion) and a hydrophilic portion (polar portion), which may be adsorbed onto a surface or an interface. Such compounds are, for example, emulsifiers and coemulsifiers.

The emulsifiers and coemulsifiers used in the composition according to the invention, when it is in emulsion form, are chosen from those conventionally used in the fields of cosmetics and dermatology. The emulsifier and the coemulsifier may be present in the composition in a proportion preferably ranging from 0.3% to 30% by weight and better still from 0.5% to 20% by weight, relative to the total weight of the composition.

O/W surfactants that may especially be mentioned include (CTFA): cetearylglucoside, PEG-40 stearate, sorbitan tristearate, sorbitan stearate, polysorbate 60, sorbitan stearate/sucrose cocoate mixture, glyceryl stearate/PEG-100 stearate mixture, PEG-400, glyceryl stearate, PEG-6/PEG-32/glycol stearate mixture. W/O surfactants that may especially be mentioned include the polyglyceryl-4 isostearate/cetyldimethicone copolyol/hexyl laurate mixture and the mineral oil/petrolatum/ozokerite/-glyceryl oleate/lanolin alcohol mixture.

The combinable composition according to the invention may also additionally comprise at least one pulverulent compound. The pulverulent compounds may be chosen from the pigments and/or nacres and/or fillers and/or mixtures thereof usually used in cosmetic compositions. Advantageously, the pulverulent compounds represent from 0.1% to 99.9% and preferably from 60% to 99.9% by weight, relative to the total weight of said composition. When said composition according to the invention is a powder, for example for the face, the pulverulent compounds preferably represent from 80% to 99.9% by weight, relative to the total weight of the composition.

The pigments may be white or colored, and mineral and/or organic. Among the mineral pigments that may be mentioned are titanium dioxide, optionally surface-treated, zirconium oxide or cerium oxide, also iron oxide or chromium oxide, manganese violet, ultramarine blue, chromium hydrate and ferric blue. Among the organic pigments that may be mentioned are carbon black, pigments of D & C type, and lakes based on cochineal carmine or on barium, strontium, calcium or aluminum.

The nacreous pigments may be chosen from white nacreous pigments such as mica coated with titanium or with bismuth oxychloride, colored nacreous pigments such as titanium mica with iron oxides, titanium mica with, especially, ferric blue or chromium oxide, titanium mica with an organic pigment of the abovementioned type, and also nacreous pigments based on bismuth oxychloride.

The fillers may be mineral or organic, and lamellar or spherical. Mention may be made of talc, mica, silica, kaolin, Nylon powders (Orgasol from Atochem or Nylon 12), poly-β-alanine powders and polyethylene powders, Teflon, bismuth oxychloride, lauroyllysine, starch, boron nitride, tetrafluoroethylene polymer powders, hollow microspheres such as Expancel (Nobel Industrie), Polytrap (Dow Corning) and silicone resin microbeads (for example Tospearls from Toshiba), precipitated calcium carbonate, magnesium carbonate, magnesium hydrocarbonate, hydroxyapatite, hollow silica microspheres (Silica Beads from Maprecos), glass or ceramic microcapsules, metal soaps derived from organic carboxylic acids containing from 8 to 22 carbon atoms and preferably from 12 to 18 carbon atoms, for example zinc, magnesium or lithium stearate, zinc laurate or magnesium myristate.

The composition may also comprise any compound usually used in such compositions, such as thickeners, antioxidants, fragrances, preserving agents, surfactants or active agents.

As cosmetic, dermatological or hygiene active agents that may be used in the composition of the invention, mention may be made of moisturizers, vitamins, essential fatty acids, sphingolipids and sunscreens. These active agents are present in an amount generally used in powders. In particular, they are present in a proportion of from 0.001 to 20% relative to the total weight of the composition.

Needless to say, a person skilled in the art will take care to select this or these optional additional compound(s), and/or the amount thereof, such that the advantageous properties of the composition according to the invention are not, or are not substantially, adversely affected by the envisioned addition.

The compositions according to the invention may be prepared in the usual manner by those skilled in the art. They may be in the form of a free or compact powder, an anhydrous gel, an aqueous gel, a W/O or O/W emulsion or a multiple emulsion. They may be, for example, in the form of a stick or dish that may be used by taking up with the finger or with a sponge, or in the form of cream: in particular, they find use as blushers, face powders, eye shadows, lip powders, care or makeup powders for the face or the body, or deodorant powders, lipsticks, foundations, mascaras, eyeliners or concealer products. They may also be in the form of a free powder, especially as face powders, care or makeup powders for the face or the body, or deodorant powders. They may also be in the form of a solution or a dispersion and may find use as nail varnishes.

These compositions for topical application may especially constitute a cosmetic composition, protective hygiene composition, medicated composition or care composition for the face, for the neck, for the hands, for the integuments or for the body (for example fragranced or unfragranced anhydrous care powder, or antisun powder), a makeup composition (for example a makeup gel) or an artificial tanning composition.

In one particular embodiment of the invention, the combinable composition according to the invention comprises at least 80% of pulverulent compounds. The product then obtained has a particular texture, in the form of a gum, i.e. a solid product, which does not become deformed under its own weight.

When the composition according to the invention is in the form of a gum, this gum may be in a dish and it may be used as a scrubbing and/or makeup composition. The product is preferably taken up by finger, by rubbing a finger several times over the surface of the gum: small gum balls thus detach and may then be applied to the skin. By rubbing these small balls on the skin, the dead cells are scrubbed from the skin, and the skin is massaged without any aggressive effect. The composition according to the invention is then used for its scrubbing properties. By this action, the colored particles contained in the composition according to the invention are also uniformly distributed. The skin is thus both soft and colored. The composition according to the invention is thus also used for making up the skin. Finally, the small balls, which are solid, are removed at the end simply by brushing the skin with the hand, and skin that is both soft and made up is thus obtained. No sticky or greasy effect is felt on the skin.

The compositions according to the invention may be used alone, in application to body or facial skin or to mucous membranes. These compositions have very good staying power and very good transfer-resistance properties once applied. Moreover, the sweat or sebum secreted by the skin interacts with the proadhesive material over time, further reinforcing the staying power of the composition and its transfer-resistance properties.

In another embodiment of the invention, the composition according to the invention is used separately and in combination with a combined composition comprising a diffusing compound.

This diffusing compound is such that it forms, when coupled with the proadhesive material, a system with adhesive properties, referred to hereinbelow and throughout the patent application as an “adhesive system”. The diffusing compound develops the bonding power of the proadhesive material by diffusing into the polymer network of said material, preferably during use on the skin, to obtain the desired cosmetic effect. It is important for the proadhesive material and the diffusing compound not to be in contact before use, within the packaging, so as not to give rise to a sticky feel. In the case of a pulverulent composition comprising said proadhesive material, there is also a risk of instability of the composition, i.e. during contact between the proadhesive material and the diffusing compound, the powders run a serious risk of becoming aggregated.

The “adhesive system” according to the invention satisfies the following conditions:

G′_{AS}(2 Hz, 35° C.)≧10³ Pa,

10⁸ Pa≧G′_{AS}(35° C.), preferably 10⁷ Pa≧G′_{AS}(35° C.), and

G′_{AS}(2×10⁻² Hz, 35° C.)≦3×10⁵ Pa,

in which:

G′_{AS}(2 Hz, 35° C.) is the elastic shear modulus of the adhesive system, measured at a frequency of 2 Hz and at a temperature of 35° C.,

G′_{AS}(35° C.) is the elastic shear modulus of the adhesive system at a temperature of 35° C.,

G′_{AS}(2×10⁻² Hz, 35° C.) is the elastic shear modulus of the adhesive system, measured at a frequency of 2×10⁻² Hz and at a temperature of 35° C.

In one preferred embodiment of the invention, the adhesive system also satisfies the following condition:

G″_{AS}/G′_{AS}(0.2 Hz, 35° C.)≧0.35,

in which:

G″_{AS}(0.2 Hz, 35° C.) is the viscous shear modulus of said adhesive system, measured at a frequency of 0.2 Hz and at a temperature of 35° C.,

G′_{AS}(0.2 Hz, 35° C.) is the elastic shear modulus of said adhesive system, measured at a frequency of 0.2 Hz and at a temperature of 35° C.

In one preferred form of the invention, the following applies:

G′_{AS}(2 Hz, 35° C.)≧10⁴ Pa.

In another preferred form of the invention, the following applies:

G′_{AS}(2×10⁻² Hz, 35° C.)≦5×10⁴ Pa.

More preferably, the adhesive systems according to the invention satisfy the following four conditions:

G′_{AS}(2 Hz, 35° C.)≧10⁴ Pa, and

10⁸ Pa≧G′_{AS}(35° C.), preferably 10⁷ Pa≧G′_{AS}(35° C.), and

G′_{AS}(2×10⁻² Hz, 35° C.)≦5×10⁴ Pa, and

G″_{AS}/G′_{AS}(0.2 Hz, 35° C.)≧0.35.

The diffusing compound may be any oil of low molecular mass, for example with a molecular mass of less than or equal to 100,000 and preferably less than or equal to 20,000. This diffusing compound may be chosen from polyisobutylenes, lanolins, aliphatic or aromatic hydrocarbon-based resins, terpenic resins and mixtures thereof, resins of aliphatic or aromatic hydrocarbon-based “tackifying resin” type; it is at least partially compatible with the proadhesive material.

As diffusing compounds that are particularly suitable for the present invention, mention may be made of polyisobutylenes with a relative molar mass of less than 150,000, such as, for example, the polyisobutylenes with respective relative molar masses Mv of 40,000, 55,000 and 85,000 sold under the respective trade names “Oppanol B 10”, “Oppanol B 12” and “Oppanol B 15” by the company BASF, polyisobutylenes with a relative molar mass Mv of less than or equal to 10,000, for instance the polyisobutylenes of molar mass 455 to 2000 sold under the trade name “Napvis” by the company BP Chemicals or the polyisobutylenes sold under the name “Parleam” by the Ets B. Rossow et Cie, or the polyisobutylenes sold under the trade name “Vistanex LM” by the company Exxon, and mixtures thereof.

These diffusing compounds are particularly suitable in combination with the preferred proadhesive materials of the invention, for instance polyisobutylenes with relative molar masses greater than 150,000 of the Oppanol range from BASF, especially the polyisobutylenes with relative molar masses Mv of 200,000, 400,000 and 1,110,000 sold under the respective trade names “Oppanol B 30 SF”, “Oppanol B 50 SF” and “Oppanol B 100” by the company BASF, and the polyisobutylenes with relative molar masses (viscosimetric mean) of between 900,000 and 2,200,000, sold under the trade name “Vistanex MM” by the company Exxon, and mixtures thereof.

Preferably, the weight ratio between the proadhesive material and the diffusing compound ranges from 0.01 to 10, more preferably from 0.05 to 2 and better still from 0.1 to 2.

Preferred “proadhesive material+diffusing compound” combinations of the invention that may also be mentioned include proadhesive materials such as block polymers of the styrene-butadiene-styrene, styrene-(ethylene-butylene)-styrene or styrene-isoprene-styrene type, for instance those sold under the trade name “Kraton” from Shell Chemical Co. or “Vector” from Exxon, combined with aliphatic or aromatic hydrocarbon-based tackifying resins, for instance the resins sold under the name “Piccotac” and “Hercotac” from Hercules or “Escorez” from Exxon.

Besides the diffusing compound, the “combined composition” may comprise any cosmetically acceptable support as defined in the present patent application for the composition according to the invention. It may also be in any of the gel, emulsion or stick forms already defined in the present patent application for the composition according to the invention.

The combination of the composition according to the invention and of the combined composition forms a kit that may be used for any care and/or makeup cosmetic product. After application of the two compositions to the skin, a cosmetic product with very good staying power and/or very good transfer-resistance properties is obtained.

The invention is illustrated in greater detail by the examples that follow.

### EXAMPLE 1

The Applicant prepared the composition in gum form below:

A) Pulverulent Phase:

| | |
|---|---|
| talc | 57.9% |
| iron oxides | 2.8% |
| Nylon powder | 10% |
| titanium oxide | 3% |
| methylsilsesquioxane resin | 5% |
| preserving agent | 0.3% |

B) Fatty Phase:

| | |
|---|---|
| polyisobutylene of molecular mass | 2% |
| 40,000 sold under the trade name | |
| “Oppanol B 10” by the company BASF | |
| polyisobutylene of molecular mass | 2% |
| 1,110,000 sold under the trade name | |
| “Oppanol B 100” by the company BASF | |
| (proadhesive material) | |
| isododecane | 16% |

The polyisobutylene of molar mass 1,110,000 in this composition has a value of G′ (35° C.)≧6×10⁴ Pa for any frequency ranging from 2×10⁻² to 2 Hz.

The composition was prepared in the following manner: the polyisobutylenes of the fatty phase were mixed in the isododecane. Next, the compounds of the pulverulent phase were mixed in a Baker mixer. The fatty phase was added dropwise. The whole was mixed again and then compacted.

A composition in the form of a gum is obtained. Small balls of this gum are taken up by rubbing the surface of the product several times with the finger. By then rubbing these small balls on the skin, the skin is both scrubbed and made up. The makeup obtained is homogeneous and uniform. The skin is particularly soft.

### EXAMPLE 2

The Applicant prepared the cosmetic base A below in the form of a W/O emulsion:

Composition A:

Phase A:

| | |
|---|---|
| sorbitan isostearate | 4.2% |
| preserving agent | 0.2% |

Phase B1:

hydrogenated isobutylene 12%

Phase B2:

| | |
|---|---|
| polyisobutylene of molecular mass | 2% |
| 1,110,000 sold under the trade name | |
| “Oppanol B 100” by the company BASF | |
| (proadhesive material) | |
| isododecane | 12% |

Phase C:

| | |
|---|---|
| water | 70.7% |
| magnesium sulfate | 0.7% |
| preserving agent | 0.2% |

The polyisobutylene of molar mass 1,110,000 in this composition has a value of G′ (35° C.)≧6×10⁴ Pa for any frequency ranging from 2×10⁻² to 2 Hz.

This emulsion was prepared according to the standard methods for preparing W/o emulsions. Phase A was first prepared by mixing. Phase B1 was then added with stirring while heating. Phase B2 is added after predissolving the polyisobutylene in the isododecane while heating. This results in an oily phase, which is heated for 5 minutes at 80° C. This mixture is cooled to 20° C. All the compounds of phase C are mixed together and then heated until the preserving agent has dissolved. Next, phase C is cooled to 20° C. and then added slowly to the mixture A+B1+B2.

The Applicant then prepared the “combined composition” B below, which is a face powder.

Composition B:

| | |
|---|---|
| acetyl tributyl citrate | 8% |
| iron oxide (red) | 5% |
| talc | 79% |
| polyisobutylene of molecular mass | 8% |
| 455 sold under the trade name | |
| “Napvis D 07” by the company BP | |
| Chemicals (diffusing compound) | |

The adhesive system consisting of the proadhesive material of composition A and the diffusing compound of the “combined composition” B in a ratio of 0.25 has the following characteristics:

G′_{AS}(2 Hz, 35° C.)=7×10³ Pa, and

G′_{AS}(2×10⁻² Hz, 35° C.)=2×10³ Pa

G″_{AS}/G′_{AS}(0.2 Hz, 35° C.)=0.35

Composition A is applied, for example to a person's face. Composition B is then applied over composition A. The makeup product obtained has noteworthy staying power and shows very little transfer.

### EXAMPLE 3

The Applicant prepared the kit below comprising:

a base i) for nails comprising the proadhesive material,

a nail varnish composition ii) containing the diffusing compound.

Base i):

polyisobutylene of molecular mass 1,110,000 sold under the trade name “Oppanol B 100” by the company BASF (proadhesive material) 5%

heptane qs 100%

The polyisobutylene of molar mass 1,110,000 in this composition has a value of G′ (35° C.)≧6×10⁴ Pa for any frequency ranging from 2×10⁻² to 2 Hz.

Nail Varnish ii):

| | | |
|---|---|---|
| nitrocellulose | | 12% |
| N-ethyl-o,p-toluenesulfonamide | | 4% |
| acetyl tributyl citrate | | 4% |
| pigment | | 1% |
| hectorite | | 1.2% |
| isopropyl alcohol | | 5.1% |
| polyisobutylene of molecular mass | | 10% |
| 455 sold under the trade name | | |
| “Napvis D 07” by the company BP | | |
| Chemicals (diffusing compound) | | |
| ethyl acetate, butyl acetate | qs | 100% |

The adhesive system consisting of the proadhesive material of the base i) and of the diffusing compound of the nail varnish ii) (“combined composition” according to the invention) in a ratio of 0.5 has the following characteristics:

G′_{AS}(2 Hz, 35° C.)=3×10⁴ Pa, and

G′_{AS}(2×10⁻² Hz, 35° C.)≦2×10⁴ Pa

The base i) is applied to the nails or to false nails. The varnish ii) is then applied. The product obtained has noteworthy staying power.

### EXAMPLE 4

A cosmetic product containing a proadhesive material that can interact with sebum to create an adhesive system that satisfies the conditions cited previously is envisioned, on the face.

The proadhesive material can belong to the family of polyisobutylenes, for instance Oppanol B100 from BASF.

This material has a value G′ (35° C.)≧6×10⁴ Pa for any frequency ranging from 2×10⁻² to 2 Hz.

The sebum is represented by artificial sebum as described in “An original procedure for quantitation of cutaneous reabsorption of sebum” by D. Blane, D. Saint-Leger, J. Bandt, S. Constans and P. Agache, arch. Dermatol. Res., 1989, 281: pp. 346-350 and cited in WO 99/59538, having the composition:

| | | |
|---|---|---|
| Palmitic acid | 5 | g |
| Squalene | 15 | g |
| Cholesterol | 6 | g |
| Oleic acid | 25 | g |
| Lauric acid | 1 | g |
| Dervacid 3155 | 3 | g |
| Myristic acid | 3 | g |
| Evening primrose oil deodorized | 34 | g |
| Glycerol isostearate | 3 | g |
| Coronet lanolin | 5 | g |
| Total = | 100 | g |

The adhesive system consisting of the proadhesive material Oppanol B100 and of the artificial sebum in a 50/50 ratio has the following characteristics:

G′_{AS}(2 Hz, 35° C.)>10⁴ Pa

G′_{AS}(2×10⁻² Hz, 35° C.)<10⁵ Pa

An emulsion foundation of water-in-oil type is prepared:

| | | |
|---|---|---|
| Polyisobutylene Oppanol B100 | | 5% |
| Isododecane | | 20% |
| Sorbitan isostearate | | 4% |
| Pigments (black, brown, etc. iron oxide) | | 10% |
| Nylon powder (Orgasol) | | 6% |
| Magnesium sulfate | | 0.7% |
| Preserving agents | | 0.2% |
| Water | qs | 100% |

The makeup product obtained has noteworthy staying power and shows little transfer.

1. A pulverulent cosmetic and/or physiological composition comprising at least one pulverulent compound and at least one proadhesive material satisfying the following condition:
10⁸ Pa≧G′ (35° C.)>2×10⁴ Pa for any frequency ranging from 2×10⁻² to 2 Hz,
in which:
G′ (35° C.) is the elastic shear modulus of said proadhesive material, measured at a temperature of 35° C.

2. The composition as claimed in claim 1, such that the pulverulent compounds are present in a content ranging from 60% to 99.9% and more preferably from 80% to 99.9% by weight, relative to the total weight of the composition.

3. The composition as claimed in either of claims 1 and 2, such that the proadhesive material is chosen from block and random copolymers comprising at least one monomer or a combination of monomers whose resultant polymer has a glass transition temperature of less than room temperature, and blends thereof.

4. The composition as claimed in one of the preceding claims, such that the proadhesive material is chosen from:
polyurethanes,
acrylic polymers,
silicones,
butyl rubbers, especially from polyisobutylenes,
ethylene/vinyl acetate polymers,
polyamides optionally modified with fatty chains,
natural rubbers,
and blends thereof.

5. The composition as claimed in the preceding claim, such that the proadhesive material is chosen from polyisobutylenes with a relative molar mass Mv of greater than or equal to 150,000.

6. The composition as claimed in any one of the preceding claims, such that the proadhesive materials are present in a content ranging from 0.1% to 99%, preferably from 0.1% to 30% and more preferably from 0.1% to 10% by weight, relative to the total weight of the composition.

7. The composition as claimed in any one of the preceding claims, such that it also comprises a cosmetically acceptable support.

8. The composition as claimed in any one of the preceding claims, such that it comprises at least one cosmetically or physiologically acceptable oil chosen especially from carbon-based, hydrocarbon-based and/or silicone-based oils of mineral, animal, plant or synthetic origin, and mixtures thereof.

9. The composition as claimed in any one of the preceding claims, such that it comprises at least one volatile oil.

10. The composition as claimed in claim 7, such that the volatile oil is present in a content ranging from 0.1% to 99% by weight, preferably from 0.1% to 60% and more preferably from 0.1% to 40%, relative to the total weight of the composition.

11. The composition as claimed in the preceding claim, such that the weight ratio of volatile oil to the proadhesive material according to the invention ranges from 1 to 20, more preferably from 1 to 10 and better still from 2 to 4.

12. The composition as claimed in the preceding claim, such that it comprises at least 80% of pulverulent compounds.

13. A makeup or care kit comprising:
i) a “combinable” composition comprising at least one proadhesive material that satisfies the following condition:
10⁸ Pa≧G′ (35° C.)≧2×10⁴ Pa for any frequency ranging from 2×10⁻² to 2 Hz, preferably:
10⁷ Pa≧G′ (35° C.)≧2×10⁴ Pa for any frequency ranging from 2×10⁻² to 2 Hz,
in which:
G′ (35° C.) is the elastic shear modulus of said proadhesive material, measured at a temperature of 35° C., and
ii) a “combined composition” comprising at least one diffusing compound such that the “proadhesive material+diffusing compound” adhesive system satisfies the following conditions:
G′_{AS}(2 Hz, 35° C.)≧10³ Pa
10⁸ Pa≧G′_{AS}(35° C.), preferably 10⁷ Pa≧G′_{AS}(35° C.), and
G′_{AS}(2×10⁻² Hz, 35° C.)<3×10⁵ Pa,
in which:
G′_{AS}(2 Hz, 35° C.) is the elastic shear modulus of the adhesive system, measured at a frequency of 2 Hz and at a temperature of 35° C.,
G′_{AS}(35° C.) is the elastic shear modulus of the adhesive system, at a temperature of 35° C., for any frequency within the range between 2×10⁻² and 2 Hz,
G′_{AS}(2×10⁻² Hz, 35° C.) is the elastic shear modulus of the adhesive system, measured at a frequency of 2×10⁻² Hz and at 35° C.

14. The kit as claimed in the preceding claim, such that the adhesive system also satisfies the following condition:
G″_{AS}/G′_{AS}(0.2 Hz, 35° C.)≧0.35,
in which:
G″_{AS}(0.2 Hz, 35° C.) is the viscous shear modulus of said adhesive material, measured at a frequency of 0.2 Hz and at a temperature of 35° C.,
G′_{AS}(0.2 Hz, 35° C.) is the elastic shear modulus of said adhesive material, measured at a frequency of 0.2 Hz and at a temperature of 35° C.

15. The kit as claimed in either of claims 13 and 14, such that:
G′_{AS}(2 Hz, 35° C.)≧10⁴ Pa.

16. The kit as claimed in one of claims 13 to 15, such that:
G′_{AS}(2×10⁻² Hz, 35° C.)≦5×10⁴ Pa.

17. The kit as claimed in one of claims 13 to 16, such that:
G′_{AS}(2 Hz, 35° C.)≧10⁴ Pa, and
10⁸ Pa≧G′_{AS}(35° C.), preferably 10⁷ Pa≧G′_{AS}(35° C.), and
G′_{AS}(2×10⁻² Hz, 35° C.)≦5×10⁴ Pa, and
G″_{AS}/G′_{AS}(0.2 Hz, 35° C.)≧0.35.

18. The kit as claimed in one of claims 13 to 17, such that the proadhesive material is chosen from block and random copolymers comprising at least one monomer or a combination of monomers whose resultant polymer has a glass transition temperature of less than room temperature, and blends thereof.

19. The kit as claimed in one of claims 13 to 18, such that the proadhesive material is chosen from:
polyurethanes,
acrylic polymers,
silicones,
butyl rubbers, especially from polyisobutylenes,
ethylene/vinyl acetate polymers,
polyamides optionally modified with fatty chains,
natural rubbers,
and blends thereof.

20. The kit as claimed in one of claims 13 to 19, such that the proadhesive material is chosen from polyisobutylenes with a relative molar mass Mv of greater than or equal to 150,000.

21. The kit as claimed in one of claims 13 to 15, such that the proadhesive materials are present in a content ranging from 0.1% to 99%, preferably from 0.1% to 30% and more preferably from 0.1% to 10% by weight, relative to the total weight of the composition.

22. The kit as claimed in one of claims 13 to 21, such that the diffusing compound is chosen from oils with a molecular mass of less than or equal to 100,000 and preferably less than or equal to 20,000.

23. The kit as claimed in the preceding claim, such that the diffusing compound is chosen from polyisobutylenes, lanolins, aliphatic or aromatic hydrocarbon-based resins, terpenic resins, and aliphatic or aromatic hydrocarbon-based tackifying resins, and mixtures thereof.

24. The kit as claimed in the preceding claim, such that the diffusing compound is chosen from polyisobutylenes with a relative molar mass of less than 150,000.

25. The kit as claimed in any one of claims 13 to 24, such that the proadhesive material is chosen from block polymers of the styrene-butadiene-styrene, styrene-(ethylene-butylene)-styrene and styrene-isoprene-styrene type, and the diffusing compound is chosen from aliphatic or aromatic hydrocarbon-based tackifying resins.

26. The kit as claimed in any one of claims 12 to 25, such that the “combined composition” comprises a cosmetically acceptable support.

27. Use, in or for the manufacture of a pulverulent cosmetic or physiological composition, of at least one proadhesive material as defined in any one of claims 1 to 12, to reduce, or even eliminate, the transfer and/or migration, and/or to increase the staying power, of the composition applied onto human lips and/or skin onto a support placed in contact with the deposit.

28. Use, in or for the manufacture of a pulverulent cosmetic or physiological composition, of at least one proadhesive material as defined in any one of claims 1 to 12, to increase the color fastness of the composition over time.

29. A nontherapeutic care and/or makeup process for human skin, mucous membranes, semimucous membranes and/or integuments, which consists in applying to said human skin, mucous membranes, semimucous membranes and/or integuments a cosmetic composition as defined in any one of claims 1 to 12.
